(19) United States
(12) Patent Application Publication
(10) Pub. No.: US 2011/0301527 A1
(43) Pub. Date: Dec. 8, 2011

(54) Moist Heat Drug Delivery Apparatus and Method

(76) Inventor: Alvin Stewart Ostrow, Atlanta, GA (US)

(21) Appl. No.: 13/154,431

(22) Filed: Jun. 6, 2011

Related U.S. Application Data
(60) Provisional application No. 61/351,289, filed on Jun. 4, 2010.

(51) Int. Cl. A61M 5/44; A61N 1/30

(52) U.S. Cl. 604/20; 604/291

(57) ABSTRACT

A novel moist steam heating cabinet unit is used as a medical device for humidification, hydration and dispensing of medicinal Hot Packs. The technology provides multifunctional options for both medicinal and non medicinal hot pack treatment as well as for other applications and uses, for example—including creation, manufacturing and curing of pharmaceutical compounds that require a controlled steam process and method. A moist heating device is a unit designed and constructed to deliver a timed, heated fluidic steam coming from a moisture generating heating source. This moist heating by employing steam heat activates the therapeutic potential of a portable topical compress or hot pack wrap applicator, of various shapes and sizes, for transdermal use and neat applications of a “wet medicinal dressing.” The moisture provides the activating liquid and fluidic environment for the flowing through, titrating, and permeating delivery of drugs or medicaments held within a medicinal pack.

### RELATED APPLICATION

This application claims priority to U.S. Provisional Patent Application No. 61/351,289 filed Jun. 4, 2010 and entitled “Medicinal Dispensing Apparatus And Method.” The complete disclosure of the above-identified priority application is hereby fully incorporated herein by reference.

### BACKGROUND

Historically over thousands of years medicinal packs have been applied as a “stupe” (defined. A hot, wet, often medicated cloth used as a compress) for topical, transdermal applications. Medicinal packs or stupes (citation here) have been patented and known in the art to become easily applied to any needed target area, providing medicinal solution(s) externally to an ailing body part for a variety of indications. Evidence in the scientific research literature that transdermal drug delivery when combined with heat increases the effectiveness of topical drug delivery. These indications include but are not limited to treatment of musculo-skeletal conditions, pain, arthritis, psoriasis, eczema, autism, stress and a variety of skin disorders. According to a paper published by Wade Hull, MS entitled Heat-Enhanced Transdermal Drug Delivery: A Survey Paper* “Much research is being done in order to find new and more effective ways to enhance the topical delivery of these drugs. Although complex chemical enhancers have been integrated into some transdermal delivery systems, physical agents such as electricity (iontophoresis), ultrasound (phono- or sonophoresis), and magnetism are becoming increasingly popular as enhancers.1 An even simpler mechanism for externally regulating transcutaneous drug absorption is the application of heat. Heat is expected to enhance the transdermal delivery of various drugs by increasing skin permeability, body fluid circulation, blood vessel wall permeability, rate-limiting membrane permeability, and drug solubility. According to Kligman, diffusion through the skin, as elsewhere, is a temperature-dependent process, so raising the skin temperature should add thermodynamic drive.5 Heat is known to increase the kinetic energy of both the drug molecules and the proteins, lipids, and carbohydrates in the cell membrane. Heating prior to or during topical application of a drug will dilate penetration pathways in the skin, increase kinetic energy and the movement of particles in the treated area, and facilitate drug absorption. Heating the skin after the topical application of a drug will increase drug absorption into the vascular network, enhancing the systemic delivery but decreasing the local delivery as the drug molecules are carried away from the local delivery site.1

Knutson et al. recently investigated the mechanisms involved in temperature-enhanced skin permeability. Results indicated that the increased skin permeability of lipophilic drugs results from temperature-induced alteration of the lipid structure, which involves the disordered arrangement of the lipid bilayer structure and its fluidization.6 Further studies indicate that temperature changes of approximately 5° C. are necessary to cause measurable changes in cell membrane permeability.1

The effect of temperature on in vitro transdermal fentanyl flux was estimated by Gupta et al using cadaver skin at controlled temperatures of 32° C. and 37° C. Over this 5-degree range, the drug flux approximately doubled. Given the doubling of release rate in vitro with a 5° C. change in temperature, an in vivo study was conducted in 20 volunteers to determine regional skin-temperature differences under occlusion. Transdermal placebo systems (10 cm2) were placed on areas of the thigh, forearm, back, chest, and postauricular areas. The results indicated that skin temperature under occlusion does not differ sufficiently from site to site to cause different drug-input rates. Gupta et al predicted that since “the diffusion process depends on the activation energy,” an increase in body temperature would increase the fentanyl permeation rate. Assuming that the diffusion rate from the delivery system remained unchanged during a 3° C. temperature increase, they predicted that the maximum serum concentration level at the middle of the 3-day application period would increase by 25% (from 2.1 to 2.6 ng/mL for a 100-mg patch).7″

Table 1 below shows examples of the use of heat with transdermal drug delivery.

TABLE 1

| | | | Percent | | |
|---|---|---|---|---|---|
| Drug | Temperature | Solubility | Increase | (° C.) | (g/100 mL) |
| Barbital | 20 | 0.629 | 138% | 37 | 0.949 |
| Phenobarbital | 20 | 0.088 | 209% | 37 | 0.184 |
| Sulfadiazine | 20 | 0.00616 | 161% | 38 | 0.0099 |
| Tolbutamide | 27 | 0.0077 | 184% | 37.5 | 0.0142 |

In the instant invention supports the theory that moist heat is more effective method for transdermal drug delivery than that of employing dry heat. In an internet publication “Iontophoresis: Maximizing Treatment Effectiveness, James R. Scifers, DScPT, PT, SCS, LAT, ATC” mentions to improve the effects of active drug delivery using iontophoresis states “Finally, for chronic conditions, apply moist heat prior to treatment in order to increase tissue permeability. Just like a hot shower opens the skin's pores, using a moist hot pack before treatment will make the skin more permeable to medication.” Up until present, a physiotherapy therapy moist hot pack treatment was delivered, provided with the commercially available Hydrocollator Units sold by Chattanooga Corporation, Chattanooga, Tenn., USA. After 60 years, Chattanooga Group still makes its original Hydrocollator stainless steel tank tub for soaking their heated “HotPac”—‘a canvas bag filled with Bentonite clay” quote here.
In current day the hydrocollator unit has not changed since its initial conception that was invented by an Athletic Trainer, Lee Jensen, in 1947. A hot water heating unit is manufactured with a steel housing container tank for holding and heating of hot packs. The hot pack unit tank is filled with water for submersing the packs underwater. An electrically heated heating element placed at the bottom of the unit tank causes the water to be heated. The hydrocollator “HotPac” moist heat packs are covered by a canvas material matrix containing within it “inert bentonite sand-like clay materials” that are placed, soaked, hydrated and heated within a heated water reservoir tank. A vertical holding tray is provided for holding the packs for submerged heating. HotPacs are made and sold in a variety of large and small sizes for different body parts. When properly heated in the tank, the packs are then taken out of the reservoir to be placed, wrapped in towels of varying thickness before being dispensed on a target body part. Wrapping the packs in towels prevents burning the patient. Hydrocollator packs are used only for the sole purpose of provided heat therapy for musculoskeletal indications. Hydrocollator current therapeutic options are the use of moist heat generated from the pack for treatment of pain, arthritis, muscle spasm and a number of indications. However, there exist no good solutions for the delivery of both pharmaceuticals, etc., applied with moist heat, especially for use in a clinic or home environment. Hydrocollator heating units have many drawbacks which are discussed herein. The steel Tank Units are clumsy and not user friendly. The tank temperature is not visibly read by the device making it difficult by the operator to easily be regulate and control. Water temperature within the Hydrocollator tank unit is approximately 160° F. +(71° C+), and the water scalding temperature is approximately 120° F. (49° C.). Tank hydrocollator water heating units do not notify the operator of the conditions within the tank in real time. The operator must manually put a thermometer in the hot water to manually record/log the unit water temperature. Hot water temperature measurement must constantly be observed by the operator to observe the heating units' daily thermal performance which is time consuming. Also there is no alarm system to alert the operator of a malfunction and may cause further liability to both practitioner and the patient. Further, the water levels in tank must be constantly replenished due to heat induced evaporation. Another drawback is when practitioners often have to inconveniently reach or bend-over the hydrocollator tank. They have to clumsily use a tong to pick-up the packs which are heavy with water and hydrated clay sand weight. Operators at times may try manually “handle the hot-packs” with their hands when the tongs are unavailable or not accessible. The procedure of taking out the Hot-Packs from the water heating unit comes with the risk of burning themselves and also creating a hot “wet mess”. Further, the tank unit can often become unsanitary and requires constant cleaning and maintenance causing potential unsanitary dangers to both practitioner and patient. When HotPacs are constantly used in a heated fluid bath environment, the HotPac canvas matrix becomes worn out. Subsequently the packs' matrix opens up and leaks out the packs' internal contents where it “leaches out” into the heating unit water tank causing a pungent, salient, unbecoming odor. Hot packs submersed underwater under these conditions in a tank opens the possibility of contamination. Therefore, this type of hot pack immersion system housed in a tank is not conducive for a clean, neat therapeutic clinical environment for both the practitioner and patient being treated. Hydrocollator heating units constantly need to be periodically drained, disinfected and cleaned, causing practitioner's loss of valuable time. The hydrocollator tank model has the following warnings from the manufacturer*: Never adjust the thermostat too high. The thermostat is extremely sensitive to the slightest adjustment.
Clean the tank periodically as described in the maintenance portion of this manual.
Always replace Hydrocollator HotPacs as soon as they show signs of wear.

If the unit is to be left unattended for a period of time, unplug the unit, remove the packs, empty the water, and clean the tank.
Do not move the Hydrocollator while filled with water. Tipping over could result in
burns to the user or others.

Always unplug the Hydrocollator from the power source before attempting to empty water from unit.

Always destroy and replace HotPacs which are exposed to contamination: hazardous or cleaning materials, bodily fluids, mold, etc.

*“Chattanooga Corporation, Instructions for the Use and Operation of the Hydrocollator M-2 Master Heating Unit. December. 1990.”

The hydrocollator system does not provide for optimum therapeutic heating effects and benefits for the patient. It does not provide for a consistent and constant source for reheating while on the patient “cools off” over time. To dispense a hot pack practitioners take the hot-packs out of the stationery tank heating unit at one location of a room and must walk with the pack toward the patient at another location in the treatment room for placement directly on the patient. The Hydrocollator tank unit and packs do not provide any alerting mechanism to inform or alert both the practitioner or the patient regarding the temperature within the pack in real time, and prevent potential injury to the patient. Further, current day hot pack devices do not conveniently allow for moist heat combined with drug delivery packs applied directly to the skin. There are problems with current options—lack of controlled treatment temperature and the inability to provide controlled moist heat for medicinal hot packs. Also, hydrocollator tank units are bulky, messy and expensive. This invention is an attempt to remedy the shortcomings of the prior art and technology.

In the process of preparing hot packs, stupes or fomentations, patients and health care practitioners had to immerse a medicinal pack by dipping, soaking the pack with into a basin or sink of hot water until the pack was fully saturated with hot water. In the practice preparing and dispensing medicinal packs, stupes or fomentations, practitioners and users risked losing and over-diluting most or all of the medicines within the pack, wasting its contents in the sink basin. This wasteful messy makeshift practice and procedure caused potential loss and effectiveness of medicinal packs or stupes purpose and their intended application.

### DETAILED DESCRIPTION OF EXEMPLARY EMBODIMENTS

The instant invention provides greater convenience and choice as an alternative to having oneself bath or immerse a body part or full body for hydrotherapy in a medicinal solution. This invention can be indicated and helpful especially for those individuals with a weak or debilitated constitution who cannot take a full body medicinal or hot salt bath treatment. There is a need for portable, user-friendly, ergonomic labor saving device to prepare and assist delivery of moist heat packs to the patient with a variety of therapeutic options in a controlled manner. In order to create a more effective, sanitary, time and labor saving moist hot pack heating device to allow medicinal drug delivery and non-medicinal hot pack treatment, the inventor by requesting letters patent shall provide the various embodiments as described herein.

Based upon US provisional filing, the instant invention involves a novel moist steam heating cabinet unit as a medical device for humidification, hydration and dispensing of medicinal Hot Packs. The technology as described herein is an advancement in the state of the art, and provides multifunctional options for both medicinal and non medicinal hot pack treatment as well as for other applications and uses, for example—including creation, manufacturing and curing of pharmaceutical compounds that require a controlled steam process and method. A moist heating device is a unit designed and constructed to deliver a timed, heated fluidic steam coming from a moisture generating heating source. This moist heating unit and method in the instant invention by employing steam heat activates the therapeutic potential of a portable topical compress or hot pack wrap applicator, of various shapes and sizes, for transdermal use and neat applications of a “wet medicinal dressing”. Said moisture provides the activating liquid and fluidic environment for the flowing through, titrating, and permeating delivery of drugs or medicaments held within a medicinal pack. A portable accessory moist heat steam pad unit is provided for continuous dispensing of moist heat to a medicinal or non-medicinal hot pack applied to a patients target area.

This invention involved the combination of therapeutics and moist heat—deliver in a controlled manner (deliver a therapeutically relevant amount of therapeutics to the skin at a therapeutically relevant rate).

The rate can be controlled by the controlled delivery of moist steam heat to the therapeutic-containing component. The combined moist heat/therapeutic will then become delivered to the skin in a controlled manner.

This instant invention provides the proper heat temperature in relationship to how much drug delivery is being delivered topically from medicinal pack. As the epidermis is exposed to moist heat temperature, the amount of drug transport through the skin incrementally increases. Increased heat causes increase drug delivery through the skin. Each drug has a different unique transdermal transport behavior across the skin in accordance to their molecular structure. It is important to understand the relationship between temperature degree of moist heat and affect of heat on the drug transport temperature mechanisms for both the drug and the skin. An LCD monitor will provide to the operator or patient a menu of options advising what temperature should be used with the medicinal or non-medicinal pack, timer for treatment, time clock, voice actuation, voice teacher regarding the device, alerts . . . etc.

The combined moist heat/therapeutic will then be delivered to the skin in a controlled manner.

The delivery of the drug can also be facilitated through the use of electrophoresis or iontophoresis on the skin. A permeable membrane of both negative and positive polarities placed on the human epidermis can be additionally put under the drug delivery medicinal “Hot-Pack”.

Hydration of drug permeating from the pack contents causes increase conductivity or the substance being delivered at C and lowers electrical impedance of the current at the dermal level which increases permeability at the Stratum Corneum. The combination of moist heat and iontophoresis is novel.

The rate of release from the pack/stupe can be controlled by modifying the properties of the matrix. The matrix can be engineered to release aqueous and drug compounds Another embodiment regarding the portable a heating pad device comprising a pack/stupe containing component and a heating component. The pack stupe is moistened and heat is applied through the heating component. The device can have a separate pack/stupe containing portion and a separate heat delivery system proximate to the pack/stupe containing portion. The pack/stupe is moistened and the pack/stupe containing portion release the moist therapeutic upon the application of heat from the heat delivery component. The pack/stupe containing portion can be engineered to provide for release of the moist heat and therapeutic when the moist heat and therapeutic are within a particular temperature range. The pack/stupe containing portion can also be engineered to release the moist heat and therapeutic at a particular release rate or range of release rates sufficient to achieve the desired therapeutic effect.

The delivery of the drug can also be facilitated through the use of electrophoresis or iontophoresis on the skin. A permeable membrane of both negative and positive polarities placed on the human epidermis can be additionally put under the drug delivery medicinal “Hot-Pack”. Hydration of drug permeating from the pack contents causes increased conductivity or the substance being delivered and lowers electrical impedance of the current at the dermal level which increases permeability at the Stratum Corneum. The combination of moist heat and iontophoresis is novel.

The rate of release from the pack/stupe can be controlled by modifying the properties of the matrix. The matrix can be engineered to release steam heat in the range of 20-43 degrees centigrade. It is the object of the present invention that a electrically powered moist steam heat cabinet medical device provides a holding area and humid environment for controlled neat sterile moist steam heat hydration of a therapeutic non-medicinal and medicinal pack. The device is be comprised of a component into which the pack/stupe/etc. is placed to prepare it for use. The device delivers moist heat to the pack/stupe/etc. until a sufficient amount of moist heat is contained in the pack/stupe. Once the desired moist heat level is reached in the cabinet unit, the pack/stupe can be applied to the skin to deliver therapeutic in a controlled manner.

Additionally, It is an object of this invention that a removable and portable accessory component is placed, attached on top of the cabinet wall or ceiling provides for continuing moist heat warming of a medicinal or non-medicinal drug treatment to a mammalian body part. The accessory device can receive distilled hot water supply from the cabinet unit.

It is an object of this device to be designed to provide the correct temperature and humidity for clean and easy dispensing of therapeutic hot pack(s). It is an object of this invention that the device is constructed for the purpose of increased effective application, utility of a medical device heating unit for the safe and effective transport and delivery of moist heat packs. It is an object that a moist heat apparatus cabinet is designed with four walls, a floor and ceiling creating a chamber; said chamber creates an open-air spaced “oven-like environment”. It is an object of this invention concerns a moist heat delivery apparatus providing multiple options in a receptacle, container or cabinet. The cabinet can be made of a heat, stain and shatter-resistant material such as plastic, fiberglass, Kevlar, stainless steel or equivalent material. The inner walls of the cabinet has multiple steam spray heads placed directionally around all sides and on the top and bottom of the cabinet to deliver steam heat into the said chamber.

It is the object of my invention to provide an improved hot pack unit dispenser with modular construction with guide rails on the internal side walls of the cabinet for conveniently holding trays, wracks, shelves, dishes for holding hot packs, stupes and fomentations within a cabinet heating chamber. Said construction and design will allow for easy dispensing of a hot packs on sliding trays or shelves to supply portable moist heat treatment to a patient. It is an object that the cabinet chamber is therefore created for placing of therapeutic hot packs or other medicinal compounds uniquely surrounded, immersed in an aqueous steam heat environment. It is an object that horizontal rows of shelves or trays with holes or other means of water permeability for allowing optimum humidification and moist heat conditions and utility for placement a hot pack or other medicinal compounds in the steam heated chamber.

The internal floor and/or walls of the moist heat apparatus are embedded with jet spray heads or nozzles that focus a steam jet spray toward the therapeutic packs surface, enabling aqueous delivery surrounding the packs placed on the shelves within the cabinet chamber.

It is an object of this invention that a placement of rows of jet spray heads on the side walls and ceiling are aimed within the cabinet chamber for the delivery of aqueous steam directionally; a heated jet spray of steam/mist is aimed at various levels and angles for a controlled hydro-saturation of the packs placed on said one or more shelves within the cabinet chamber. It is an object of this invention when the steam jets are activated, the medicinal pack(s) and their drug contents becomes hydrated, primed for drug delivery applications.

It is an object of this invention that one or more rows of mobile vertical sliding shelves or trays, or dishes are held in place by guide rails allowing for easy access, ejection of, reaching and handling of the portable therapeutic packs or optionally pharmaceutical compounds in a container resting on same trays, shelves or dish; said tray or dish or shelves can be manually or mechanically ejected from the cabinet unit for easy dispensing of items placed on the tray, dish or shelve.

It is an object of this invention to prevent the practitioner from contaminating the unit and self while preventing inappropriate handling and potential burning of the operator. It is an object of this invention that steam heat cabinet apparatus device is manufactured with prefabricated parts having easy to assemble modular components. It is an object of this invention that to continue the therapeutic treatment from a medicinal or non-medicinal hot pack, a portable steam heating accessory attached to top of the unit can be easily and removed, dislodged for portably providing continuous moist steam heat to be placed to rest directly on top of a medicinal or non-medicinal hot pack.

It is an object this invention that in addition to manufacture of the said medical device for industrial and clinical use, a small home model for one or two pack dispenser can be manufactured for home use.

It is a further object of my invention to provide a hot pack unit with a modular prefabricated construction wherein the water are contained within a tray shelf or cradle pan which becomes heated to dispense steam heat. The removable basket pan has handles on the sides, where the said steam unit can be removed without having the operator/practitioner placing his or her hands directly while removing the pack being housed on the apertured bottom wall of the basket or cradle. It is also an object of this invention that the moist heat unit can be readily formed and constructed of relatively few parts which may be easily manufactured at relatively low cost.

It is also an object of my invention to provide a multifunctional moist hot pack dispenser for dispensing regular “off-the-shelf” commercially available hot-packs as well as medicinal packs. In accordance with one of the aspects of this invention, a portable moist steam heat dispenser includes a housing having a hollow generally vertical rear wall, a generally horizontal stand portion and an overhanging top wall which extends generally horizontally from an upper portion of the vertical rear wall. A water dispensing reservoir is positioned on the vertical wall a substantial distance above the lower stand portion of the housing and a collecting pan for hot water is positioned on the stand at one side of the cabinet apparatus. A hot water outlet is provided in the overhanging top wall directly above the basket collecting and at one side of the hot pack dispenser. A superior removable hot water basket pan is provided with an outwardly extending square lip at its upper portion so that it may be supported thereby. This aspect provides hot steam with a heat monitor is designed to be the portable steam heating unit that can be placed on top of a hot pack.

An inferior collecting pan on the bottom of the cabinet floor recycles and re-circulates water back into the vertical rear wall. Should the water reservoir in the rear wall become insufficient or empty to operate the device, a monitor warning signal will go off to notify the operator that the water levels are dangerously way too low. This prevents or guards against rapid loss of water and moisture in the unit.

It is also necessary to have a device where the water is distilled and sterilized within the unit to prevent outside unwanted contamination. It is an object of this invention that the portable component supplies moist heat on top of a medicinal pack allowing hot water and medication(s) contents to move using gravity in the downward direction toward the skin, to allow moist heat and heated water to course through a medicinal pack, enabling leaching out of the combined pack contents traveling toward the direction, destination and contact with the mammalian skin for medicinal and/or physiotherapy applications.

The device requires sufficient moisture and dew level and should this level be insufficient, an LCD monitor/alarm system will notify and warn the operator. A glass front door can allow the practitioner to view outside the conditions inside the cabinet.

Pharmaceutical compounds have a specific moist heat dew point humidity and temperature where it reaches a optimum point for hydration/water solubility where they can be able to become transdermally transported. This specific dew point and humidity can also have industrial applications in the creation of certain drug compounds that require a moist heat steam environment for their curing and manufacturing process as well as increasing their water solubility. Since each drug or pharmaceutical compound has specific heating, dew and humidity point, the temperature in the cabinet must be controlled as to not cause any degrading, destruction or change of chemical compounds produced for their specific intended therapeutic purpose.

Therefore, a safe method is provided for a safe steam moist heating cabinet for controlling the amount of moist steam heat. Water dripping in a water feeder is turned into steam and disturbed via jets or nozzles. Thus generated steam in the cabinet chamber is discharged through the steam channels which keeps the inside of the cabinet connected to the outside of the cabinet. The temperature of steam passing through the steam channel is measured by a steam temperature measuring device. The amount of water fed from the water feeder is controlled in response to the measured temperature.

The steam cabinet device will have fans located on the side walls strategically placed so that temperature can be controlled for even cooling in the chamber. Further, fans assist in directionally focusing the steam mist toward the target pack or medicinal product lying on the shelf and create a unified consistence temperature in the chamber.

It is an object of this invention that the accessory attachment to the cabinet to provide continuous steam heating of packs unit with its own electrical outlet for or port or can optionally be battery operated. It is an object of the portable steam unit to have a flexible heating pad-like design; said design has latches on the outer borders for holding and heating of said medicinal and non medicinal hot packs.

It is an object of this invention that within the heading pad, a system or mesh of flexible and malleable heating wires that are heated by electric means are encased in a water proof flexible material matrix in the first superior layer of the portable steam heating pad accessory unit. The heating component wiring has separation plate prevents seepage and interaction with the bladder and treatment area to avoid and prevent electroshock and burning.

Said material matrix forms a safe barrier preventing electroshock between the electrical heating aspect and the fluid dispensing aspect of the pack. Said accessory moist heat or steam heating pad is made of a hypoallergenic and heat and fire resistant material made of fiberglass, pyrofoil (ADL ISULAFLEX, Inc. Ontario, Canada) that holds, surrounds the heating pad forming a “matrix” that creates a pad, allowing the packaging, holding and dispensing of said moist steam heat or steam heat vapors.

Said pack has a contact barrier material matrix that becomes in contact with a medicinal pack is comprised of hypoallergenic polymer permeable and/or semi-permeable membrane heat resistant and hypoallergenic materials allowing passive release of its contents in a slow, timed constant rate of permeation titrating through the barrier matrix to provide a desired rate of transdermal delivery of said medicaments in the medicinal pack below. Release of said bladder heated water through the contact barrier matrix contents activates, releases and transports a fluidic medium. It is an object that the accessory applicator heating pad unit can be made various shapes and sizes that can be directly placed on top of a medicinal or non-medicinal hot pack that can be wrapped around any desired anatomical body part.

It is an object of this invention that the second layer below the top layer of the electrical heating component of the pad is separated by a water proof non-electrical conducting material matrix, comprising of a bladder holding a water supply for the creation of aqueous steam heating. The bladder water supply is connected to a water outlet which can be attached, hooked-up by an attachable hose via a cap opening in the bladder for filling. The device in the instant invention is designed to conveniently irrigate and slowly control filling the bladder reservoir with the proper amount of water from a water tap with distilled hot water supplied by the console unit. Said bladder contains a network of multiple porous supply tubing delivers the fluid medium are responsible for regulating slow dispersion, irrigation and titration of the fluid contents contained within the bladder reservoir via a system of branching ducts, allowing it contents to seep through the inferior aspect. The bladder layer in the present invention is located superiorly on top of, being placed above the packs' medicinal contents. The bladder layer is designed to release the fluidic contents within the reservoir to become in direct contact with the pack to allow moist heat to slowly seep through, permeate and hydrate a medicinal or non-medicinal hot pack.

It is an object of this invention that A porous layer of the heating pad accessory is inferior to the bladder for slow leakage and seepage of moist steam heat. It is an object of this invention that this said accessory heating pad device increases the inventions' utility and purpose, created to enhance the ability of portability of providing enhanced moist steam heating methods externally when medicinal or non-medicinal packs are not stored in the stationary console model. Through warming of the bladder by the electric component above, its application on the dermis allows increases stratum corneum and epidermal permeability, facilitating increased penetration of the medicaments being delivered topically. Warming or heating of the water contents enhances gradual release of said medicinal contents through the pack matrix. Therefore the present accessory hot-pack heating pad accessory dispensing system will provide a predetermined controlled rate for dosage, timed titration and permeation of its contents as desired or prescribed by the patients' physician or pharmacist for greater efficiency and accuracy.

It is an object that the moist heat pad will provide a timer and temperature gauge with an alert system to assist the patient and the operator in providing the correct temperature and timing for delivering the appropriate therapeutic treatment. It is an object that the moist heat steam heating pad accessory will have sensors that can be connected to a medicinal or non-medicinal hot pack or surface of the patients skin to create a temperature loop systems for maintaining a desired temperature for therapy and drug delivery. Hot packs need to be can be dispensed with controlled temperature and do not require being wrapped in a thick toweling coverage to prevent burning the patient.

It is a principal object of the invention of the heat pad accessory to provide a semi-permeable membrane that the contact layer of the moist heating pad that is with a pack or skin. This membrane can deliver the moist heat from the bladder reservoir. Said membrane is non-irritating to the skin while administering a moist heated fluid or alternatively passive or active delivery of a broad range of drugs.

In accordance with a preferred embodiment of the invention there is provided a combined moist heating pad system for delivery of moist heat and a pharmaceutical medium comprising: a layer of permeable foam for retaining therein a quantity of pharmaceutical medium exhibiting at least partial ionization; and an electroconductive membrane disposed under said foam layer through which the an aqueous solution or pharmaceutical medium passes, by passive or active transport through said membrane to provide moist heat and drug treatment therapy via the skin.

In a preferred embodiment of the present invention the transdermal drug delivery system is provided as an unlinked patch device with an electroosmotic, electroporous membrane comprised of an inert biochemical substance which becomes actively electroosmotic when an electric charge is passed through the membrane. During iontophoresis, an electric current is passed into the membrane, and it becomes electro-bioactive enabling drug solution to be passed through the electrode membrane wall to a target area on the skin surface creating an electroporative effect.

The dermal contact surface of the membrane is constructed out of substances close to the structure of human skin, based on the membranes used in artificial kidney membranes such as sulfonated polysulfone (PSF), so as to be non-irritating. This will reduce skin rejection of polymers currently used to accomplish transdermal transport of drug solutions. Alternatively, any commercially available transdermal membrane can be used with the contact surface membrane (for example, IonClad R1010, R1030, R4010 or R4030 from Pall Corp., Port Washington, N.Y.).

The bladder reservoir can be additionally provided with foam layer placed on top of the membrane for controlled microtitration of aqueous or drug solutions.

In another embodiment of the present invention the patch is provided with a biosensor for sensing when the drug or water is no longer present in the bladder reservoir alerting the patient or physician via an audible or visual signal.

It is an object that temperature of a stupe, fomentation or pack from the console unit must be maintained for continuance of optimum therapy conditions for either drug delivery or physiotherapy.

An embodiment of how this can be accomplished in a portable component of the console model for commercial and home use is the placement of a heated bladder reservoir allowing for permeating slow timed steam/moist heat above a pack, fomentation or stupe for delivering consistent therapeutic temperature. The top aspect of the console can become detached as is known as the top bladder layer providing for a portable accessory moist drug delivery heating pad.

Approaches to drug delivery include (PG pack), however, these approaches do not allow for the controlled delivery of the therapeutic.

The technology disclosed in this patent application provides a solution to the controlled delivery of therapeutics and moist heat.

One embodiment comprises a moist heat delivery component that is placed proximate to a pack/stupe/etc., wherein the moist heat delivery component releases moist heat in manner sufficient to result in the precise titration of therapeutic agent from the pack/stupe/etc. to the skin.

And; precise rate of moisture permeating through the pack which determines the rate of drug permeating to the skin.

The moist heat delivery component will release moist heat at a rate sufficient to result in the controlled release of therapeutically relevant amount of drug from the pack/stupe/etc.

In the instant invention as described in this patent herein is an innovation in the field of drug delivery solving problems of application, utility and portability. It is designed as an effective time saving applicator system to alleviate the excessive fuss, guess work and clumsy application of creating “home-made” packs. Medicinal mineral or Epsom Salt Pack applications in a pack, stupe, or fomentation in the present invention can be easily applied to any needed target area, effectively providing controlled topical delivery of medicinal solutions externally to an ailing body part or area. The present invention has the ability to control, regulate the dosage and rate of what is being dissolved, evacuated, transported, absorbed or released from the medicinal pack through its barrier matrix. . The rate of release can be controlled by gravity, vacuum, or other force acting on the device.

The novel invention is to create a device that will be able to provide moist heat to medicinal hot packs that will provide drug and medicament delivery, however, can also be used for providing moist heat to non-medicinal drug delivery packs.

The invention is a device to deliver moist heat and therapeutics. The moist heat and therapeutics can be A is mixing with B at controlled rate in a hydrous compound to go to C.

The device will provide for the controlled delivery of moist heat and therapeutic compounds to the skin. Safety—temperature sensing component—sensing temperature at least one location of skin, therapeutic pack, and moist heat delivery component.
alert user/practitioner when desired temperature range is reached
alert user/practitioner of unsafe temperature

When the pack is activated via hydration, its contents cross through the material matrix creating passive transdermal drug delivery stupe to the surrounding dermis being in direct contact with said pack applicator.

The medicinal pack or stupe applicator is topically and easily applied for slow, timed, and controlled release of its contents for allowing a gradual slow “oozing out” its medicinal contents via the material matrix to relieve the affected area. When said pack applicator cools off or its' contents is released and used up, another applicator pack, stupe and fomentation product can be employed to repeat the treatment procedure as many times as desired or needed.

1. A moist steam heat cabinet apparatus, comprising:
a cabinet having a floor, sides, and an inner lining;
an electrically powered moist steam heat cabinet medical device provides a holding area and humid environment for controlled neat sterile moist steam heat hydration of a therapeutic non-medicinal -and medicinal pack, said moist heat cabinet having a multiple arrangement of steam spray nozzles around said sides and floor of said inner lining of said cabinet, said cabinet being of various shapes and sizes, said device delivering moist heat to prepare hot packs/stupes/fomentation until a sufficient amount of moist heat is contained in the pack/stupe at a correct temperature and is ready for topical dispensing to a mammalian or human body part.

2. A moist steam heat cabinet apparatus as in claim 1, having controlled temperature exhibited on a display temperature control heating display board, said controlled temperature being pre-determined according to the drug's susceptibility and ability to be optimally activated thermally and delivered transdermally, said controlled temperature exhibition alerting a practitioner of pack temperature within said cabinet.

3. A moist steam heat cabinet apparatus as in claim 1, wherein a controlled spray mist of water fluid hydrates drug delivery packs with a multiple mini-series of aligned jets along the length of the pack, wherein spray jets on the side walls are designed to deliver exact amounts of heat moisture “with a spray mist ejection” to allow the total areas of the pack to become hydrated and heated to the desired temperature without loss or over-leaching out of the drug contents.

4. A moist steam heat cabinet apparatus as in claim 1, further comprising a sliding shelf or tray with levels and horizontal rows of open spaced water permeable holding shelves for the holding and storage of the packs.

5. A moist steam heat cabinet apparatus as in claim 4, wherein the sliding shelf or tray comprises an ejectable sliding shelf or tray that can be optionally removed manually with pre-manufactured handles for easy removal of the shelf or tray while holding/dispensing of its contents.

6. A moist steam heat cabinet apparatus as in claim 1, further comprising an electronic indicator that indicates that the drug delivery packs within said cabinet and placed on the shelf or tray are ready for use.

7. A moist steam heat cabinet apparatus as in claim 1, wherein the spray jets provide the appropriate timed moisture for each pack placed in the cabinet placed in rows horizontally, obliquely, or vertically.

8. A moist steam heat cabinet apparatus as in claim 1, wherein subdivided placement of the packs arranged individually in rows allows moisture to slowly titrate to the packs without loss of their contents.

9. A moist steam heat cabinet apparatus as in claim 1, wherein the apparatus is self-cleaning.

10. (canceled)

11. A moist steam heat cabinet apparatus as in claim 1, wherein the apparatus is portable for home use or is a stationary console for office use.

12. (canceled)

13. (canceled)

14. A moist steam heat cabinet apparatus as in claim 1, wherein the apparatus comprises a specific spray mist setting for regular hydrocollator packs.

15. A moist steam heat cabinet apparatus as in claim 1, wherein the apparatus comprises a specific spray mist setting for medicinal packs.

16. A moist steam heat cabinet apparatus as in claim 1, wherein the apparatus comprises a specific temperature for hydrocollator packs

17. A moist steam heat cabinet apparatus as in claim 1, wherein the apparatus comprises a specific temperature for medicinal packs according to the drug employed.

18. A moist steam heat cabinet apparatus as in claim 1, wherein transport and permeation of drugs are accomplished by at least one of gravity, vacuum pressure, and iontophoresis.

19. A moist steam heat cabinet apparatus as in claim 1, further comprising a portable, removable moist heating pad that that creates steam and that is placed on top of a pack, the pad for alternate transfer from the apparatus and placement for direct contact on the human skin or over a medicinal or non-medicinal pack, to maintain continuous steam heat to a medicinal or non-medicinal hot pack, the pad comprising latches to hold a pack securely below the pad to allow clean and neat placement of the device with the pack to a patient.

20. A moist steam heat cabinet apparatus as in claim 1, wherein the pack is used with FDA approved electrotherapy generators that provide direct and galvanic currents to produce Iontophoresis, electroporation, and electrophoretic treatments.

21. A moist steam heat cabinet apparatus as in claim 1, further comprising a clam-shell or strapping method to hold a pack, stupe, or fomentation in place for easy, stable usage and placement.

22. A moist steam heat cabinet apparatus as in claim 1, further comprising an electric moist heating pad accessory device that provides moist steam heating currents to electrodes that contact with an aqueous solution for physiotherapy or iontophoresis treatment.

23. A moist steam heat cabinet apparatus as in claim 1, wherein the portable moist heat system is applied to a skin surface of a human user, and wherein the human skin maintains a temperature of less than about 43 degrees Celsius during the transfer of moist heat.

23. A moist steam heat cabinet apparatus as in claim 1, wherein the apparatus produces steam hydration with a temperature ranging from 20-200 degrees Fahrenheit.
